(19) United States
(12) Patent Application Publication
(10) Pub. No.: US 2016/0067091 A1
(43) Pub. Date: Mar. 10, 2016

(54) SURGICAL HAND PIECE FOR CATARACT REMOVAL

(71) Applicant: Alcon Pharmaceuticals Ltd., Fribourg (CH)

(72) Inventors: Trent Wells, Los Angeles, CA (US); Amir Kashani, Royal Oak, MI (US); Matthew McCormick, Yucaipa, CA (US); Mark Humayun, Glendale, CA (US)

(21) Appl. No.: 14/609,291

(22) Filed: Jan. 29, 2015

Related U.S. Application Data
(60) Provisional application No. 62/045,932, filed on Sep. 4, 2014.

(51) Int. Cl. A61F 9/007

(52) U.S. Cl. CPC A61F 9/00763

(57) ABSTRACT

The disclosure herein provides systems and devices for mechanically cutting and removing lens tissue or cataract from an eye of a patient without use of ultrasonic energy.

### RELATED APPLICATIONS

This application claims the benefit of U.S. Provisional Application No. 62/045,932, filed on Sep. 4, 2014, titled “SURGICAL HAND PIECE FOR CATARACT REMOVAL,” which is hereby incorporated by reference herein in its entirety, including specifically but not limited to the devices, systems, and methods directed to a surgical hand piece.

### BACKGROUND

1. Field

The disclosure relates generally to the field of minimally invasive surgery, and more particularly, to the field of ophthalmic surgery, specifically cataract removal using a surgical hand piece.

2. Description

Various types and procedures of ophthalmic surgery often involve manipulation and/or removal of eye tissue. For example, removal and replacement of the lens is generally required for cataract surgery. After removing the natural lens during cataract surgery, an artificial lens or intraocular lens implant can then be implanted within the eye of the patient to restore and/or improve vision. Other ophthalmic surgical procedures can also require the removal of lens tissue and/or manipulation of other types of eye tissue.

With the development of new technologies in ophthalmic surgery, it is possible to effectively remove lens tissue from the eye with progressively smaller incisions which is often referred to as minimally invasive surgery. When removing lens tissue from an eye of a patient, it can also be important to avoid collateral damage to adjacent structures of the eye. For example, it is generally disadvantageous to puncture and/or otherwise damage the lens capsule of an eye of a patient while removing lens tissue from the patient. Accordingly, it can be advantageous to have a system to effectively remove lens tissue while preventing damage of other tissues of the eye during cataract removal.

### SUMMARY

Advancements in technology make it possible to use a surgical hand piece to mechanically remove a cataract from an eye of a patient without the need for ultrasonic energy, thereby reducing any or substantially all risks related to use of an ultrasonic device.

In accordance with one aspect, a device for performing ophthalmic surgery for cataract removal can comprise a casing, one or more inner assemblies can comprise, one or more device tips, and one or more inner housings and an outer housing. In some embodiments, the device wherein the one or more inner assemblies comprise a rotating element configured to rotate during use of the device. In some embodiments, the device wherein the one or more inner assemblies are configured to be coupled to a motor to produce rotational motion.

In some embodiments, the device wherein the device is used for cataract removal during ophthalmic surgical procedures. In some embodiments, the device wherein the device is used to manipulate, cut, fragment, or remove various eye tissues. In some embodiments, the device wherein the one or more device tips comprise smooth edges configured to contact a surface of a patient's eye. In some embodiments, the device wherein a portion of the one or more inner assemblies is longitudinally surrounded by the outer housing along a length of the one or more inner assemblies. In some embodiments, the device wherein the portion of the one or more inner assemblies surrounded by the outer housing is configured to rotate within the outer housing. In some embodiments, the device wherein a proximal end of the one or more inner assemblies is configured to be coupled to a distal end of the casing.

In some embodiments, the device further comprises an aspiration tube and aspiration pathway configured to be in fluid communication with a vacuum source, wherein the vacuum source can aspirate fragmented tissue from a patient's eye. In some embodiments, the device further comprises an infusion tube and an infusion pathway configured to provide an infusion of fluid into a surgical area.

In some embodiments, the device wherein the one or more device tips comprise a biocompatible metal. In some embodiments, the device wherein the one or more device tips comprise a material of a smooth texture to minimize traction with surrounding tissues. In some embodiments, the device wherein the material of the one or more device tips comprises a coating on the surface of the one or more device tips. In some embodiments, the device wherein the one or more device tips comprise a loop configuration. In some embodiments, the device wherein the loop configuration comprises more than one loop. In some embodiments, the device wherein the loop configuration comprises an off-center loop or non-symmetrical loop shape. In some embodiments, the device wherein the one or more device tips comprise a forcep configuration.

In some embodiments, the device wherein the motor is configured to drive the one or more inner assemblies in one direction, two directions, or rotate back and forth in an oscillation motion. In some embodiments, the device wherein the one or more inner housings comprise a flat strip, wherein the flat strip comprises metal twisted into helical flutes. In some embodiments, the device wherein the one or more inner housings comprise a hollow rod. In some embodiments, the device wherein the one or more inner housings comprise a twisted tube. In some embodiments, the device wherein a proximal end of the outer housing is fixed to a distal end of the casing. In some embodiments, the device wherein the one or more inner assemblies are configured to translate axially and rotate simultaneously for removal of cataracts.

In some embodiments, the device wherein the one or more inner housings comprise varying temperatures, wherein the temperature of the one or more inner housings comprise between 0° C. and 50° C. In some embodiments, the device wherein the one or more inner housings are configured to be coupled to a heat and/or cooling source configured to vary the temperature of the one or more inner housings. In some embodiments, the device wherein the one or more inner housings are configured to be heated and/or cooled during operation to cause further breakdown of the tissue and allow for easier removal.

In some embodiments, the device further comprising a mechanical mechanism for extending and retracting the one or more inner assemblies. In some embodiments, the device wherein the one or more inner assemblies are configured to be coupled to device configured to generate rotational movement, wherein the device configured to generate rotational movement is pneumatically, hydraulically, electromechanically, or mechanically driven.

For purposes of this summary, certain aspects, advantages, and novel features of the invention are described herein. It is to be understood that not necessarily all such advantages may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein.

### BRIEF DESCRIPTION OF THE DRAWINGS

FIG. 1 illustrates an overall anatomy of an eye and a schematic representation of a surgical hand piece for cataract removal.

FIG. 2 illustrates an overview of an example of one embodiment of a distal end of a surgical hand piece for cataract removal.

FIG. 3 illustrates an example of embodiments of an aspiration pathway of a surgical hand piece for cataract removal.

FIG. 4 illustrates an example of embodiments of an infusion pathway of a surgical hand piece for cataract removal.

FIG. 5A-5B illustrates an example of embodiments of an inner assembly of a surgical hand piece for cataract removal.

FIGS. 6A-6B illustrates examples of embodiments of an extended device tip and/or inner housing of a surgical hand piece for cataract removal.

FIGS. 7A-7B illustrates examples of embodiments of a retracted device tip and/or inner housing of a surgical hand piece for cataract removal.

FIGS. 8A-8B illustrates examples of embodiments of a mechanical mechanism for extending and retracting the device tip and/or inner housing of a surgical hand piece for cataract removal.

FIGS. 9A-9B illustrates examples of embodiments of a casing of a surgical hand piece for cataract removal.

FIGS. 10A-10C illustrates examples of embodiments of a surgical hand piece for cataract removal.

### DETAILED DESCRIPTION

Although several embodiments, examples and illustrations are disclosed below, it will be understood by those of ordinary skill in the art that the invention described herein extends beyond the specifically disclosed embodiments, examples and illustrations and includes other uses of the invention and obvious modifications and equivalents thereof. Embodiments of the invention are described with reference to the accompanying figures, wherein like numerals refer to like elements throughout. The terminology used in the description presented herein is not intended to be interpreted in any limited or restrictive manner simply because it is being used in conjunction with a detailed description of certain specific embodiments of the invention. In addition, embodiments of the invention can comprise several novel features and no single feature is solely responsible for its desirable attributes or is essential to practicing the inventions herein described.

The disclosure herein provides embodiments of a surgical hand piece for cataract removal during an ophthalmic surgical procedure. Some embodiments of a surgical hand piece for cataract removal described herein can effectively remove a cataract without puncturing and/or damaging the lens capsule and/or other portions of the eye. In some embodiments the device tip does not comprise any sharp edges. The sharp edges greatly reduce the operational safety of the device which detracts from one of the main advantages and uniqueness of the surgical hand piece. The device tip comprises smooth edges configured to contact the surface of the patient's eye. A surgical hand piece may comprise a casing, one or more device tips, one or more inner housings, outer housing, and/or a mechanical mechanism for extending and retracting the inner assembly. An inner assembly may comprise one or more device tips and one or more inner housings. In some embodiments, an inner assembly may comprise a rotating element configured to rotate during use of the surgical hand piece.

In some embodiments, one or more surgical hand pieces can be used during an ophthalmic surgical procedure in order to manipulate, cut, fragment, and/or remove various eye tissues. For example, a device tip or surgical hand piece can be used to manipulate, cut, fragment, and/or remove lens fragments, vitreous, or the like. Generally, during cataract removal, such device tips or surgical hand pieces can be used to break up and remove lens tissue. Embodiments of a device tip or surgical hand piece for cataract removal described herein can generally be applied during cataract surgery in order to fragment the cataract into smaller pieces for removal. However, it should be understood that embodiments of a device tip or surgical hand piece described herein can also be used in other fields and applications, such as removing cartilage, muscle, ligament, tendon, or bone tissue during surgery.

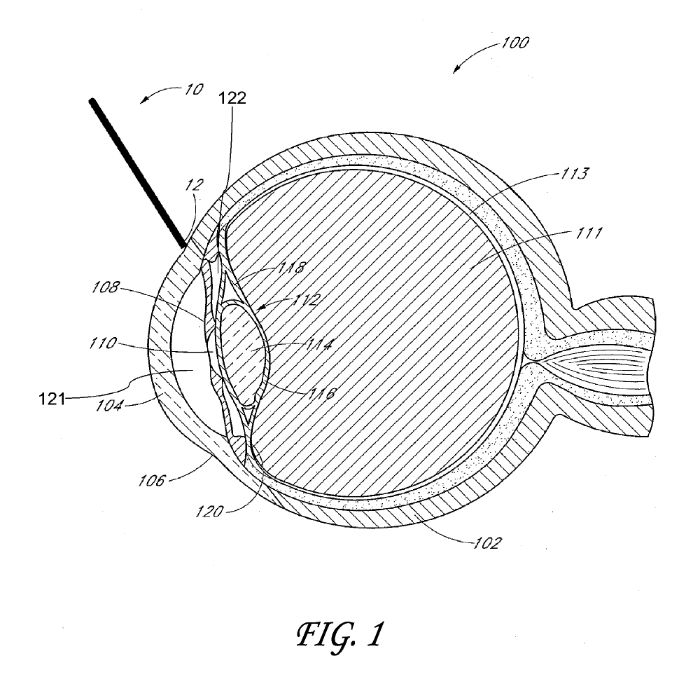

FIG. 1 illustrates a cross section of the overall anatomy of an eye 100 and a schematic representation of a distal end of a surgical hand piece for cataract removal 10. In some embodiments, a distal end of a surgical hand piece for cataract removal 10 may comprise one or more device tips, one or more inner housings, and/or an outer housing. As illustrated in FIG. 1, the sclera 102 and cornea 104 makeup the outermost layer of the eye. The sclera 102 and cornea 104 meet at the cornea/sclera junction or limbus 106. The iris 108, which is visible through the cornea 104, forms the outer diameter of the pupil 110 or opening in the iris 108. The space between the iris anterior surface of the iris and the posterior surface of the cornea is filled with fluid (aqueous humor) and called the anterior chamber 121. The lens 112 is located behind the iris 108 and pupil 110. The lens 112 comprises multiple lens fibers 114, which are surrounded by the capsule 116. The capsule 116 is a thin transparent membrane. Zonular suspensory ligaments (or zonules) 118, which are connected to the ciliary body 120, hold the lens 112 in place. The space between the posterior surface of the iris and the zonules is the posterior chamber 122. The vitreous humor or body 111, a clear gel, fills the space between the lens 112 and the retina 113 of the eye.

The lens 112 is a transparent, biconvex structure that helps to refract light to be focused on the retina 113. By changing its shape, the lens effectively changes the focal distance of the eye to allow for a sharp real image of the object of interest to be formed on the retina.

Cataracts are one of the most common ailments of the lens and result in opacity or cloudiness of the lens. Some cataracts that are advanced enough can eventually block sufficient light to obstruct the vision of a patient and may require surgery. For example, cataract surgery requires removal of the cataract and replacement of the natural lens 112 with a prosthetic lens.

In order to remove cataracts, a surgical hand piece can be used in some embodiments. The surgical hand piece can be inserted into the eye for cataract removal at one or more locations. For example, in some embodiments, a surgeon or other medical professional can make an external corneal, or corneoscleral incision of roughly 1.5 mm-3.0 mm. Next the anterior chamber 121 can be infused with a high viscosity gel, fluid or other similar substance in order to prevent the anterior chamber 121 from collapsing after efflux of the aqueous humor. Through a dilated pupil, the surgeon or other medical professional can then remove a circular area of approximately 5 mm in diameter from the capsule and insert a distal end 12 of a surgical hand piece into the anterior chamber through the external incision. The surgical hand piece can be configured to aspirate out lens material and/or cataract material, allowing the surgeon or other medical professional to implant a new lens into an eye of the patient. In the most ideal embodiment of this procedure, it is performed without damage to the adjacent ocular structures including but not limited to the sclera, corneal, iris, peripheral capsule, posterior capsule, zonules, vitreous cavity, or retina.

The surgical hand piece 10 can comprise a distal end 12 to be inserted through the external incision located at the corneal/scleral junction 106. The distal end 12 can comprised a device tip, portions of the inner housing, and/or portions of the outer housing. In certain embodiments, insertion of the surgical hand piece 10 into the eye 100 can occur in other locations as well, including near the corneal/scleral junction 106, through the sclera 102, through the cornea 104, or at other locations of the eye. Once inserted into the eye, the surgical hand piece can be used to break up cataract and/or lens tissue into smaller fragments for removal.

While breaking up the lens of an eye of a patient into smaller fragments, it can be important for the surgical hand piece not to puncture and/or otherwise damage other portions of the eye, such as the remaining capsule. In some embodiments, the surgical hand piece can comprise an ultrasonic source and/or device to assist breaking up the lens tissue. However, in such embodiments, ultrasonic damage to the corneal endothelium, capsule, iris and/or other portions of the eye is possible.

Accordingly, in certain embodiments, a surgical hand piece for cataract removal does not comprise an ultrasound energy source, but rather comprises parts of purely mechanical nature. Due to the mechanical nature of such surgical hand pieces, the safety hazards associated with ultrasonic devices are no longer an issue. For example, ultrasonic damage to the corneal endothelium, capsule, and/or other portions of the eye are no longer an issue for such mechanical surgical hand pieces. Further, by negating the need for additional ultrasonic power and thereby removing the effect of ultrasonic energy introduced into the anterior chamber 121, the risk of burning or otherwise harming the tissue within the anterior chamber 121 or around the incision site can be greatly reduced. With this added safety, a mechanical surgical hand piece for cataract removal can be used to assist cortex removal and/or polishing the capsule after cataract removal.

Further, some embodiments of a mechanical surgical hand piece for cataract removal are as effective as ultrasonic devices in terms of the time required for cataract removal. In some embodiments, the time required to remove cataract from an eye of a patient using an embodiment of a mechanical surgical hand piece is about 150%, about 140%, about 130%, about 120%, about 110%, about 100%, about 90%, about 80%, about 70%, about 60%, or about 50% of the time required to remove cataract from an eye using an ultrasonic device. In certain embodiments, the time required to remove cataract from an eye of a patient using an embodiment of a mechanical surgical hand piece compared to using an ultrasonic device is within a range defined by two of the aforementioned values.

Moreover, certain embodiments of a mechanical surgical hand piece for cataract removal are as effective as ultrasonic devices in terms of the size of the broken up cataract or lens tissue. For example, in some embodiments, the size of broken up cataract or lens tissue using an embodiment of a mechanical surgical hand piece can be about 100%, about 90%, about 80%, about 70%, about 60%, about 50%, about 40%, about 30%, about 20%, about 10%, or about 1% of the size of broken up cataract or lens tissue using an ultrasonic device. In certain embodiments, the size of broken up cataract or lens tissue using an embodiment of a mechanical surgical hand piece compared to using an ultrasonic device is within a range defined by two of the aforementioned values.

In addition, some embodiments of a mechanical surgical hand piece are cheaper to build and are more profitable for manufacturers and/or distributors. At the same time, there is less risk associated with using such mechanical surgical hand pieces to a surgeon and/or other medical professional compared to ultrasonic devices because there is less chance of ultrasonic damage to adjacent eye tissues. Lastly, because there is less risk of damaging other portions of the eye during cataract removal, using such mechanical surgical hand pieces is also advantageous for the patient, because the patient can enjoy a better outcome with greatly reduced risk of a ruptured capsule.

### Overview

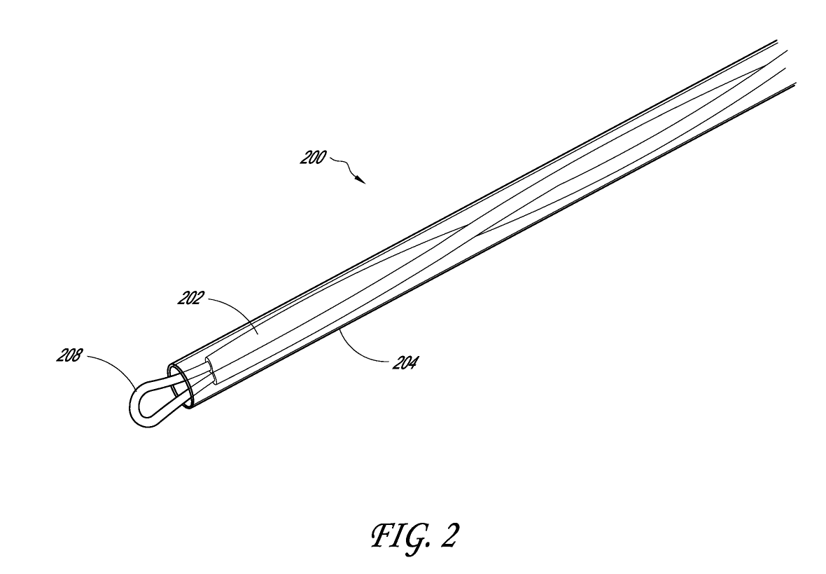

FIG. 2 illustrates an overview of one embodiment of the distal end of a surgical hand piece for cataract removal. As illustrated in FIG. 2, some embodiments of a surgical hand piece 200 for cataract removal comprise one or more inner assemblies 202, 208. In certain embodiments, a surgical hand piece 200 for cataract removal can comprise an outer housing 204 and/or an inner assembly 202, 208. In some embodiments, the one or more inner assemblies 202, 208 or a portion thereof can be longitudinally surrounded or otherwise located within an outer housing 204 along the length of the inner assembly 202, 208. In certain embodiments, a surgical hand piece 200 for cataract removal can comprise a casing 206. The outer housing 204 can be configured to surround the inner assembly 202, 208 and can be affixed to the casing 206 with axial alignment to the inner assembly 202, 208. Proximal ends of the outer housing 204 and/or inner assembly 202, 208 can further be coupled to a casing 206. In some embodiments, the proximal end of the one or more inner assemblies is configured to be coupled to a distal end of the casing. In some embodiments, the proximal end of the outer housing is configured to be coupled to a distal end of the casing.

In some embodiments, the outer housing 204 and/or inner assembly 202, 208 or portions thereof can be configured to be inserted into an eye of a patient. The inner assembly 202, 208 can further be coupled to a mechanism for rotary motion causing the inner assembly 202, 208 to rotate, thereby breaking up the lens tissue or cataract into smaller fragments for effective removal. For example, an ophthalmic surgeon and/or other trained professional can insert the outer housing 204 and/or inner assembly 202, 208 or portions thereof into an eye of a patient and turn on the device such that the inner assembly 202, 208 rotates while a portion of the inner assembly 202, 208 is within the eye of a patient in order to break up the lens tissue or cataract. Further, in certain embodiments, the inner assembly 202, 208 can be configured to translate axially and rotate simultaneously for efficient removal of cataracts.

In some embodiments, the inner assembly 202, 208 when rotating can serve to fractionate the lens material or cataract and facilitate aspiration. In certain embodiments, the inner assembly 202, 208 can serve a secondary purpose to assist aspiration similar to a screw pump.

In some embodiments, a distal end of an inner assembly 202, 208 comprises a device tip 208 configured to break up lens tissue or cataract into smaller fragments whether or not it is actually rotating. A proximal end of an inner assembly 202, 208 can be configured to be coupled to a motor or other device configured to produce rotational motion. The motor or other device can be configured to rotate the inner assembly 202, 208 in one or more directions at one or more speeds. The inner assembly 202, 208 or a portion thereof can be configured to rotate within the outer housing 204. In some embodiments, a portion of the inner assembly 202, 208 can protrude beyond the outer housing 204, and thereby rotate outside of the outer housing 204. In other embodiments, the inner assembly 202, 208 need not be rotating to perform a function. In some embodiments, the inner assembly 202, 208 can be extended outside the housing and used in static (non-rotating) position to manipulate ocular tissue.

Further, in some embodiments, the device tip 208 does not comprise any sharp edges. Accordingly, it is less likely to puncture and/or otherwise damage the capsule, even if the device tip 208 is physically contacting the capsule in a rotating or non-rotating state.

Because some embodiments of the surgical hand piece are purely mechanical in nature and do not use any ultrasonic energy there is significantly less risk of puncturing or otherwise damaging the capsule compared to using ultrasonic devices as described previously. For example, in certain embodiments, purely mechanical forces from rotation of the inner assembly 202, 208 break up the lens tissue and/or cataract. In some embodiments, the inner assembly 202, 208 can physically contact the capsule without harming the capsule 116 tissue, because the rotating movement follows the contour of the capsule 116 only and does not puncture or tear the capsule 116. In other words, in some embodiments, rotational movement of the inner assembly 202, 208 is a safer method for removal as compared to ultrasonic devices.

In certain embodiments, the surgical hand piece is configured to rotate the inner assembly 202, 208 at high speeds sufficient to prevent the lens from spinning during fragmenting of the cataract. In addition, in some embodiments, despite the rotational movement of the inner assembly 202, 208, the rotational movement does not cause the capsule to spin around and destroy the lens, because the suspensory ligaments prevent the lens/lens capsule from spinning due to the instrument motion. In fact, in certain embodiments, the lens stays stationary while a rotational movement of the inner assembly 202, 208 is applied on an eye of a patient. More specifically, in some embodiments, the inner assembly 202, 208 can fracture the cataract into small fragments without causing the lens to spin and/or the capsule to tear by separating the cataract and lens prior to applying rotational movement thereby allowing the inner assembly 202, 208 to manipulate the cataract such that the rotational movement does not force the lens to spin or cause the capsule to tear. Further, in certain embodiments, the inner assembly 202, 208 is configured to effectively stabilize the cataract at higher speeds and prevent spinning. In addition, in certain embodiments, one or more additional inner assemblies 202, 208 and/or surgical hand pieces are configured for manipulation to effectively stabilize the cataract and prevent spinning.

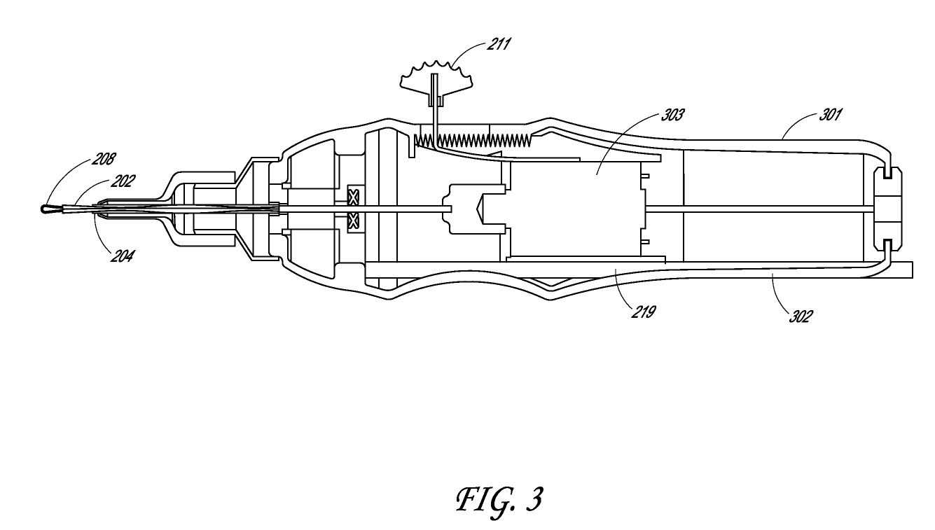

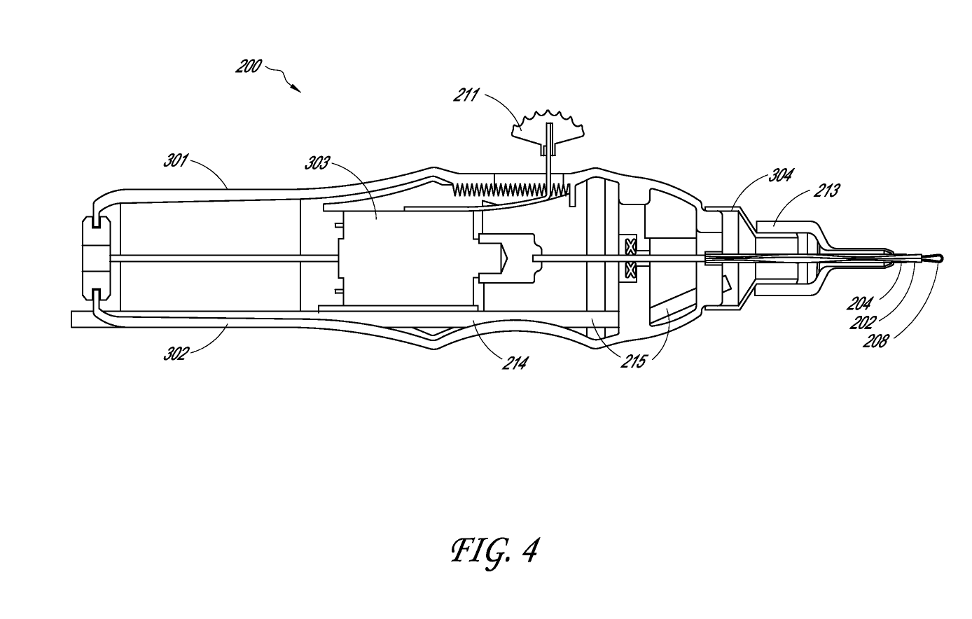

FIGS. 3 and 4 illustrate cross-sections of the surgical hand piece. The surgical hand piece can include a mechanical mechanism 211 which allows for control of the device tip length between an extended position and a retracted position as described in further detail herein. In some embodiments, the surgical hand piece does not include a mechanical mechanism or mechanism for extending and retracting the device tip. In some embodiments, the device tip can be retained or fixed in the extended position. For example, during a surgical procedure, the incision may be large enough to accommodate the device tip and the need to retract the device tip may not be necessary.

The surgical hand piece can comprises an upper hand piece 301 and a lower hand piece 302. The upper hand piece 301 and the lower hand piece 302 of the casing 206 can engage and/or lock together and remain in place during use of the surgical hand piece. A motor 303 positioned within in the surgical hand piece can be utilized to actuate or move the device tip 208 between the retracted and extended position. In some embodiments, the motor 303 or other device that provides movement of the inner assembly 202, 208 can be positioned outside of the surgical hand piece casing 206 and can be in communication with the inner assembly. The motor 303 can provide the rotary motion required to turn, rotate, or otherwise move the device tip 208. The device tip 208 and inner housing 202 are shown in FIG. 4 in the extended position and protruding from the outer housing 204.

FIG. 3 illustrates examples of embodiments of a surgical hand piece 200 comprising an aspiration tube and/or an aspiration pathway 219. FIG. 3 illustrates a cross-section of the surgical hand piece showing an aspiration pathway 219. In certain embodiments, a surgical hand piece 200 for cataract removal comprises a vacuum source. The vacuum source can be connected to a fluidic connection in some embodiments. For example, an aspiration tube and aspiration pathway are configured to be in fluid communication with a vacuum source, wherein the vacuum source can aspirate fragmented tissue from the patient's eye. Accordingly, after the inner assembly 202, 208 is used to break the cataract or lens tissue into smaller pieces, the vacuum can aspirate the fragmented pieces, thereby removing the cataract and/or lens tissue from the eye of a patient. In some embodiments, a surgical hand piece 200 for cataract removal comprises an aspiration tube and/or an aspiration pathway configured to provide the vacuum to the surgical area. For example, a distal end of the aspiration tube and/or an aspiration pathway can comprise an opening to aspirate fragments of lens tissue or cataract, while a proximal end of the aspiration tube and/or an aspiration pathway can be coupled to a vacuum pump or other means for creating a vacuum. The surgical hand piece device 200 can aspirate tissue and/or fluids while cutting or fragmenting the tissue of the eye of a patient. In some embodiments, the surgical hand piece device 200 can aspirate tissue and/or fluids even when cutting is disabled. For example, the inner assembly 202, 208 can be disabled or movement of the inner assembly 202, 208 can be stopped and the aspiration tube and/or aspiration pathway can remain on and allowing tissue and fluids to be aspirated from the surgical area.

FIG. 4 illustrates examples of embodiments of a surgical hand piece 200 comprising an infusion tube 214 and an infusion pathway 215. Further, in some embodiments, a surgical hand piece 200 for cataract removal comprises an infusion tube 214 and/or an infusion pathway 215 for providing infusion fluid into the surgical area. The infusion tube 214 and/or an infusion pathway 215 can provide infusion fluid into the surgical area during cataract removal in order to aid the removal of broken up fragments of cataract or lens tissue and prevent collapse of the anterior chamber, lens capsule, or eye in general. For example, in certain embodiments, after an inner assembly 202, 208 breaks up a cataract or lens tissue into smaller fragments, infusion fluid that is provided via the surgical hand piece can assist removal of such fragments by positioning of the fragments closer to the aspiration tube and/or an aspiration pathway. In some embodiments, the infusion pathway 215 terminates at the distal end of the surgical hand piece and can comprise an opening to provide infusion fluid to a surgical area, while a proximal end of the infusion pathway 215 is coupled to an infusion fluid source.

### Device Tip

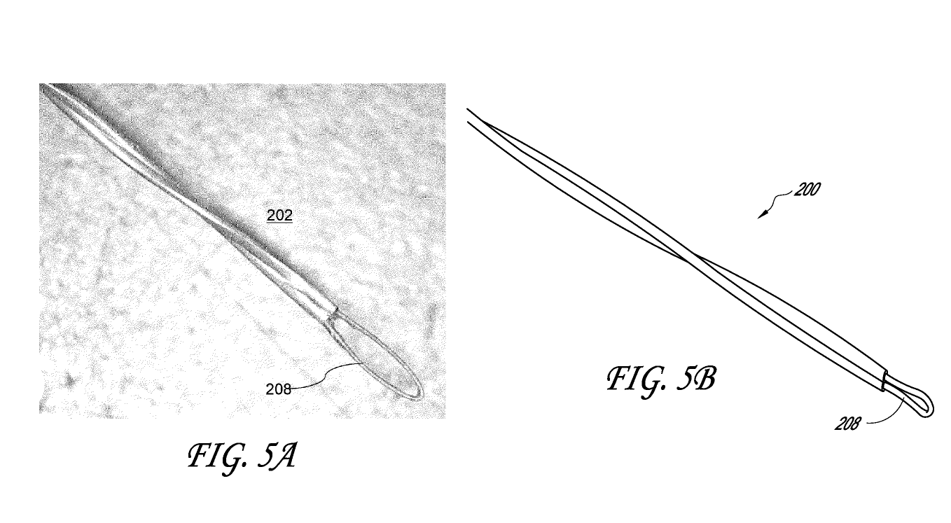

FIGS. 5A-B illustrates examples of embodiments of an inner assembly 202, 208 comprising an inner housing 202 and/or a device tip 208. FIG. 5A is a prototype of the inner housing 202 with a helical flute as the device tip 208. FIG. 5B illustrates a model of the inner housing 202 with a helical flute as the device tip 208. In some embodiments, a surgical hand piece 200 for cataract removal comprises a device tip 208 coupled to or is part of a distal end of an inner housing 202. The device tip 208 can be configured to rotate, thereby using the mechanical force generated from the rotation to break up lens tissue or cataract into smaller fragments for removal.

When the inner assembly 202, 208 is rotating, the device tip 208 can be configured to rotate and break up lens tissue or cataract into smaller fragments. The device tip 208 of an inner assembly 202, 208 can comprise a material that is sufficiently hard to break up soft lens tissue and hard cataracts. For example, in some embodiments, the device tip 208 of an inner assembly 202, 208 can be made of stainless steel or any other biocompatible metal. In some embodiments, the device tip 208 comprises material of a smooth texture to minimize traction with surrounding tissue. The smooth texture can be provided by a material coating on the surface of the one or more device tips. In some embodiments, the material coating can include a Teflon-like surface coating on the device tip. In some embodiments, the material of the device tip itself can provide the smooth texture of the device tip.

When the surgical hand piece 200 for cataract removal is not rotating, the device tip 208 and/or the inner assembly 202, 208 can further be utilized as a simple manipulation tool, reducing the need for a secondary instrument. For example, the device tip 208 and/or the inner assembly 202, 208 can be used in a similar fashion to a Drysdale for lens manipulation or a capsule polisher.

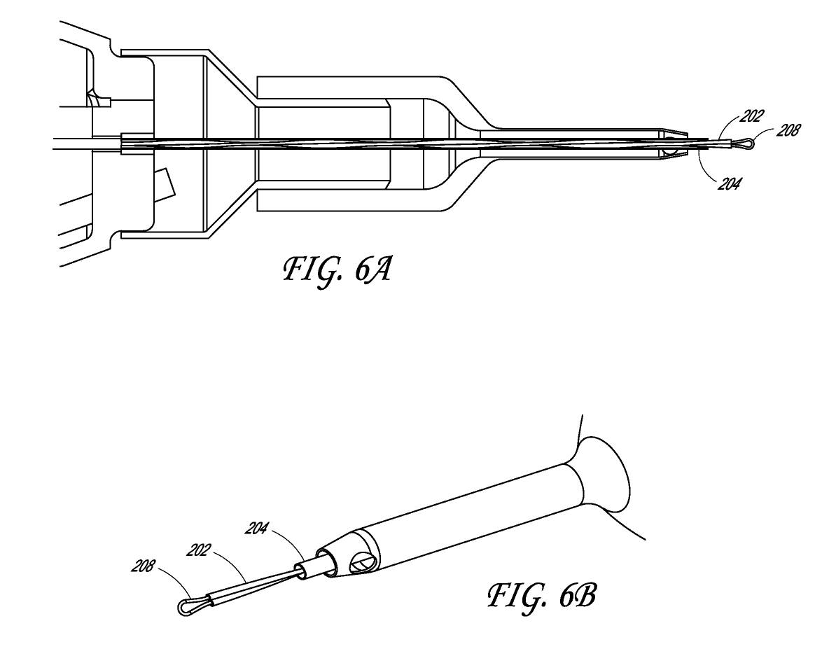

FIGS. 6A-B illustrate examples of embodiments of an inner assembly 202, 208 extended so that the device tip 208 and/or the inner housing 202 protrudes beyond the outer housing 204. FIG. 6A illustrates an enlarged section of the instrument or surgical hand piece tip. The device tip 208 is in the extended position protruding out of the outer housing 204. FIG. 6B illustrates an embodiment of an enlarged view of the extended device tip. In some embodiments, the device tip 208 and the inner housing 202 are encased in an outer housing 204. In some embodiments, the device tip 208 or a portion thereof protrudes beyond a tube or outer housing 204 that longitudinally surrounds the majority of the length of the inner housing 202. In certain embodiments, only the device tip 208 and/or the inner housing 202 or a portion thereof that extends beyond the tube or outer housing 204 can be configured to be inserted into an eye of a patient for tissue removal. In other embodiments, the device tip 208, the outer housing 204, and/or the inner housing 202 or a portion thereof can be configured to be inserted into an eye of a patient for tissue removal.

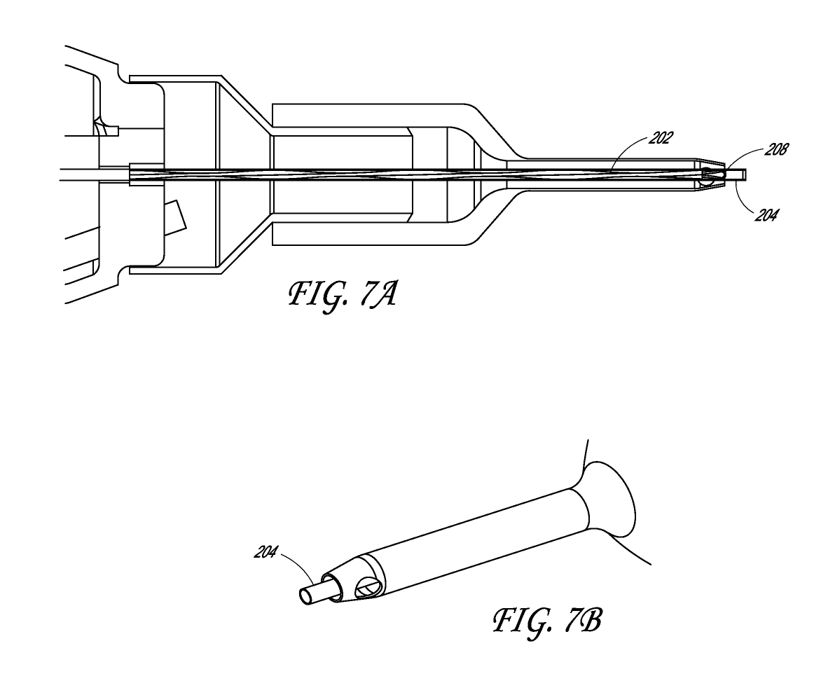

FIGS. 7A-B illustrate examples of embodiments of an inner housing 202 retracted so that the device tip 208 and/or the inner housing 202 are retracted and contained within the outer housing 204. FIG. 7A illustrates an enlarged cross-section of the surgical hand piece with the device tip 208 retracted. FIG. 7B illustrates an enlarged view of the device tip 208 retracted. In certain embodiments, the device tip 208 can be varied in diameter by adjusting the length of exposed wire or by varying the protruding length exposed at the distal tip of the outer housing 204. For example, in some embodiments, a surgical hand piece 200 comprises a mechanism for axially moving the inner assembly 202, 208 within the tube or outer housing 204, thereby varying the protruding length exposed at the distal tip of the tube or outer housing 204.

In certain embodiments, the inner assembly 202, 208 can be fashioned from a material that is designed to change shape, size, temperature, color and/or magnetic properties to achieve a surgical result.

### Device Tip—Loop Configuration

In certain embodiments, the device tip 208 can comprise one or more loops. FIGS. 5A-5B illustrate an embodiment of a device tip 208 comprising a loop. The one or more device tips can comprise a loop configuration. The loop configuration can include more than one loop. The one or more loops can be on one device tip. In some embodiments, the loop configuration can be an off-center loop or non-symmetrical loop shape. In other embodiments, the loop configuration can be a symmetrical loop shape. The one or more loops can comprise a small gage wire. In other embodiments, the one or more loops are cutout or stamped from the inner assembly 202, 208. The one or more loops can contact surrounding tissue, including but not limited to the lens capsule, iris, and cornea with minimal damage while effectively fragmenting up lens tissue or cataracts into sufficiently small fragments for removal.

In some embodiments, the device tip 208 can comprise one loop, two loops, three loops, four loops, five loops, or multiple loops. In certain embodiments, an inner assembly 202, 208 with a larger wire size comprises relatively less number of loops. In contrast, an inner assembly 202, 208 of a smaller wire size can comprise relatively more loops.

In certain embodiments, the size of the one or more loops can be varied in diameter for grabbing and/or crushing the lens. One or more loops of varying sizes can be used in connection with a surgical hand piece 200 for cataract removal. For example, in some embodiments, the radius of one or more loops can be slightly larger than the diameter of a tube or the outer housing 204 surrounding the inner housing 202 such that a constant contact is made between the one or more loops and the tube or outer housing 204 during operation. In certain embodiments, the diameter of one or more loops can be about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm, about 3.0 mm, or within a range defined by any of the two aforementioned diameters.

### Device Tip—Forcep Configuration

In some embodiments, the device tip 208 can comprise a forcep configuration. In other words, the distal tip of the inner housing 202 can terminate in forcep-like ends that can assist in grasping fragments of material, including but not limited to lens tissue or cataract. In certain embodiments, the forcep-like ends are configured to open and/or close. For example, the forcep-like ends can be configured to open as a working length of the inner housing 202 is increased or as the inner housing 202 is pushed axially into the surgical area. The forcep-like ends can be configured to close to grasp lens fragments as the working length of the inner housing 202 is decreased or as the inner housing 202 is pulled axially out of the surgical area. The forcep like tip can be extended and retracted form the inner housing in the same manner as described herein with reference to the loop configuration or the device tip 208.

### Inner Housing

In some embodiments, a surgical hand piece 200 for cataract removal comprises an inner housing 202. The inner housing 202 can be configured to rotate and thereby use the mechanical force from the rotational movement to break up cataract or lens tissue. In some embodiments, the inner housing 202 can be surrounded by a tube or outer housing 204 along a longitudinal direction. In some embodiments, the inner housing 202 can be surrounded by and/or encased in a tube or outer housing 204 along all or portions of the longitudinal direction of the inner housing 202 including the device tip 208. In some embodiments, the inner housing 202 can be surrounded by a tube or outer housing 204 along a longitudinal direction.

In certain embodiments, a distal end of an inner housing 202 can be configured to be connected to or otherwise coupled to a device tip 208 of one or more types described herein. Further, a proximal end of an inner housing 202 can be mechanically coupled to a motor or other device configured to produce rotational movement. The motor 303 or other device can be located within a casing 206 as illustrated in FIG. 4. In some embodiments, the inner housing 202 can facilitate the removal of material, including but not limited to lens tissue or cataract, by creating a conveyance mechanism that assists aspiration. In certain embodiments, the inner housing 202 can be configured to rotate at a rotational speed of about 2,000 rpm, about 2,500 rpm, about 3,000 rpm, about 3,500 rpm, about 4,000 rpm, about 4,500 rpm, about 5,000 rpm, about 5,500 rpm, about 6,000 rpm, about 6,500 rpm, about 7,000 rpm, about 7,500 rpm, about 8,000 rpm, about 8,500 rpm, about 9,000 rpm, about 9,500 rpm, about 10,000 rpm, or within a range defined by two of the aforementioned rotational speeds. Generally, the efficiency of tissue removal via the surgical hand piece 200 initially increases as the rotational speed is increased but the safety can decrease beyond a particular rotational speed.

In certain embodiments, the inner housing 202 is axially surrounded or located within an outer housing 204 along the majority of the length of the inner housing 202. The outer housing 204 can comprise a close running fit along the majority of the length of the inner housing 202 to provide support and protect the surrounding tissue during operation once inserted into the eye. In other words, the inner housing 202 can comprise a diameter that is slightly less than that of a concentric tube or outer housing 204.

In certain embodiments, the inner housing 202 can comprise varying temperatures. In some embodiments, the inner housing 202 is coupled to a heat and/or cooling source configured to vary the temperature of the inner housing 202. For example, in some embodiments, the inner housing 202 can be heated and/or cooled during operation to cause further breakdown of the tissue and allow for easier removal. In certain embodiments, the temperature of the inner housing 202 can be varied between 0° C. and 50° C. a portion thereof. In some embodiments, the inner assembly 202, 208 including both the inner housing and the device tip can comprise varying temperatures. In some embodiments, the inner assembly 202, 208 is coupled to a heat and/or cooling source configured to vary the temperature of the inner assembly 202, 208. The inner assembly 202, 208 can be heated and/or cooled during operation to cause further breakdown of the tissue and allow for easier removal. In certain embodiments, the temperature of the inner assembly 202, 208 can be varied between 0° C. and 50° C. a portion thereof.

In some embodiments, the inner housing 202 comprises varying angles or pitch along the length of the inner housing 202. In other words, in some embodiments, the inner housing 202 does not assume a fixed pitch angle along the length. In certain embodiments, the inner housing 202 may be coupled to a controller configured to vary the angle and/or pitch of the inner housing 202. For example, the angle formed between the inner housing 202 and a perpendicular axis of insertion of the surgical hand piece 200 into an eye of a patient can be or can be varied to about 5°, about 10°, about 15°, about 20°, about 25°, about 30°, about 35°, about 40°, about 45°, about 50°, about 55°, about 60°, about 65°, about 70°, about 75°, about 80°, about 85°, about 90°, or within a range defined by two of the aforementioned values.

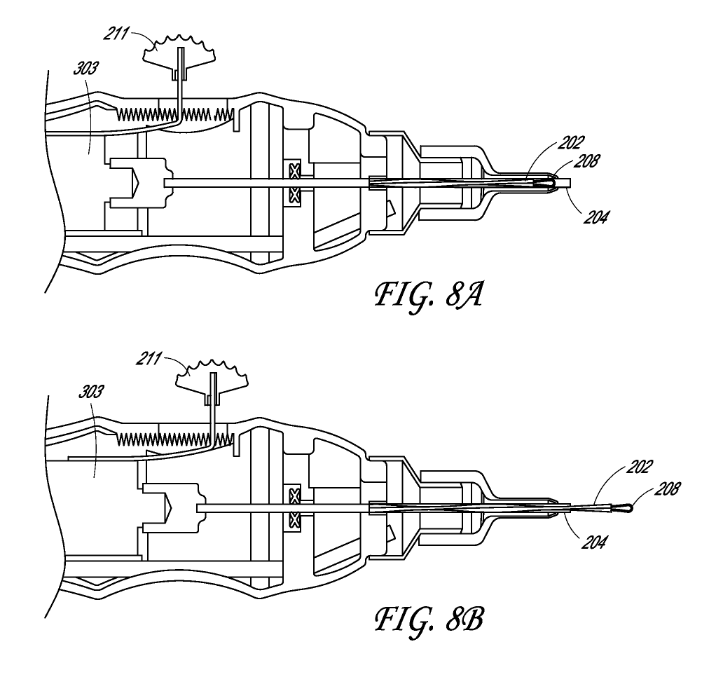

FIGS. 8A-B illustrate examples of embodiments of an inner housing 202 retracted and extended so that the device tip 208 and/or other portions of the inner housing 202 are retracted and contained within the outer housing 204 and extended and protruding outside the outer housing 204. FIG. 8A illustrates the device tip 208 retracted and FIG. 8B illustrates the device tip 208 extended. In certain embodiments, a protruding length of the inner housing 202 extending beyond the length of a tube or outer housing 204 surrounding the inner housing 202 can be varied. FIGS. 8A-8B illustrate a cross-section of a portion of the surgical hand piece displaying the inner housing 202 and device tip within the surgical hand piece apparatus. As illustrated in FIGS. 8A-8B, in some embodiments, the protruding length can be varied directly by the operator actuation of a mechanical mechanism 211 causing movement of the entire drive system axially along the surgical hand piece 200 as described herein in more detail.

For example, the mechanical mechanism 211 can be actuated by an electromechanical system which receives electrical input from the operator allowing the inner housing 202 to be varied indirectly. Also, the mechanical mechanism 211 can be a linear slide directly connected to the drive system to allow forward and backward movement of the assembly along the axis of the surgical hand piece 200 thus extending and retracting the inner housing 202. In certain embodiments, the mechanical mechanism 211 can be a rack and pinion directly connected to the drive system to allow forward and backward movement along the axis of the surgical hand piece 200 thus extending and retracting the inner housing 202. In some embodiments, the mechanical mechanism 211 can be a screw directly connected to the drive system to allow forward and backward movement of the assembly along the axis of the surgical hand piece 200 thus extending and retracting the inner housing 202. In some embodiments, the surgical hand piece 200 comprises a control system incorporated with one or more mechanical mechanisms 211 described herein to vary the length of the inner housing 202 in an oscillatory manner at different frequencies as determined by the operator.

In certain embodiments, the surface texture of the inner housing 202 can be varied systematically. For example, similar to varying grit sizes of sand paper, the inner housing 202 can comprise one or more abrading materials of varying surface texture. In some embodiments, the surface of the inner housing 202 can be textured in order to increase traction and remove material more quickly.

In certain embodiments the inner housing can be fashioned from a material that is designed to change shape, size, temperature, color and/or magnetic properties to achieve a surgical result.

### Inner Housing—Solid Rod/Strip Configuration

In some embodiments, the one or more device tips 208 can be connected to a long, narrow shim or flat strip. In some embodiments, the long, narrow shim or flat strip can be the inner housing. In some embodiments, the long, narrow shim or flat strip can be positioned within the inner housing. The long, narrow shim or flat strip can be made of the same or different material as the device tip 208 located at a distal end of the shim or flat strip. For example, the shim or flat strip can be made of stainless steel or any other material. In some embodiments, the device tip 208 at a distal end of an inner assembly 202, 208 can be welded to the shim or flat strip. The shim or flat strip or portions thereof can comprise a straight or curved configuration. In other embodiments, the shim or portions thereof can comprise a screwed configuration or spiral configuration. For example, the shim or flat strip can comprise metal twisted into helical flutes of substantially equal or varying pitch along the majority of the length or longitudinal axis.

In some embodiments, the shim or flat strip, the device tip 208, or portions thereof can be placed inside an outer housing 204. In certain embodiments, the shim or flat strip and/or the device tip 208 or portions thereof can move axially within the housing 204. In certain embodiments, a working length of a shim, flat strip, and/or device tip 208 can be varied by a control system at predetermined oscillating frequencies.

The proximal end of the shim can be coupled to a motor or other device configured to generate rotational movement in some embodiments. The motor or other device, in certain embodiments, can drive in one direction, two directions, and/or rotate back and forth in an oscillation motion.

In some embodiments, the long, narrow shim or flat strip comprises a flat piece that is twisted to create the screwed, spiral configuration. Due to the spiral configuration, in some embodiments, the long, narrow shim or flat strip can generate an aspiration force and/or suction force when it rotates. In some embodiments, the long, narrow shim or flat strip can generate a pressure gradient for aspiration when it rotates. Accordingly, lens tissue which is grinded by the device tip 208 can be removed from the eye via such aspiration.

In certain embodiments, a surgical hand piece 200 for cataract removal comprises a vacuum tube. In certain embodiments, a surgical hand piece 200 for cataract removal comprises a fluidic connection to a vacuum source. A proximal end of the fluidic connection can be coupled to a vacuum source configured to provide aspiration. In some embodiments, a proximal end of the fluidic connection located opposite the distal end of a surgical hand piece 12 can be coupled to a vacuum source to provide aspiration for removal of tissue material that is fragmented by the device tip 208. A distal end of the fluidic connection can comprise an opening. Accordingly, the vacuum source can be transmitted along the fluidic pathway through the distal opening, thereby removing broken up cataract or lens tissue. In some embodiments, the vacuum source and the rotational movement of a twisted shim or flat strip, in combination, assist removal of broken up cataract or lens tissue.

### Inner Housing—Tube Configuration

In some embodiments, a device tip 208 at a distal end of an inner assembly 202, 208 can be coupled to a hollow rod or tube. In some embodiments, the hollow rod or tube can be an inner housing. The hollow rod or tube can be made of the same or different material as the device tip 208. For example, the hollow rod or tube can be made of stainless steel or any other material. In some embodiments, the device tip 208 at a distal end of an inner assembly 202, 208 can be welded to a hollow rod or tube. The hollow rod or tube or portions thereof can comprise a straight or curved configuration.

The proximal end of the hollow rod or tube can be coupled to a motor or other device configured to generate rotational movement in some embodiments. The motor or other device, in certain embodiments, can drive in one direction, two directions, and/or rotate back and forth in an oscillatory motion.

Furthermore, in certain embodiments, a proximal end of the hollow rod and/or tube is coupled to a vacuum source to provide suction for removal of tissue material that is fragmented by the device tip 208. In some embodiments, a distal end of the hollow rod and/or tube coupled to the device tip 208 can comprise an opening. Accordingly, the vacuum source can be coupled to a fluidic connection to the hollow rod and/or tube through the distal opening. Lens tissue or cataract that is fragmented by the device tip 208 can be removed from the surgical area via aspiration.

In some embodiments, the hollow rod or tube, the device tip 208, or portions thereof can be placed inside an outer housing 204. In certain embodiments, the hollow rod or tube and/or the device tip or portions thereof can move axially within the outer housing 204. In certain embodiments, a working length of a hollow rod or tube and/or device tip 208 can be varied by a control system at predetermined oscillating frequencies. For example, the working length can extend about 0 mm, about 1 mm, about 2 mm, about 3 mm, about 4 mm, about 5 mm, about 6 mm, about 7 mm, about 8 mm, about 9 mm, about 10 mm, and/or between a range defined by two of the aforementioned lengths.

### Inner Housing—Twisted Configuration

In certain embodiments, a device tip 208 at a distal end of an inner assembly 202, 208 is coupled to or connected to a tube that is twisted. In some embodiments, the inner housing can be the twisted tube. The twisted tube can be made of the same or different material as the device tip 208. For example, the twisted tube can be made of stainless steel or any other biocompatible material. In some embodiments, the device tip 208 at a distal end of an inner assembly 202, 208 can be welded to a twisted tube. The twisted tube can comprise a screw configuration or spiral configuration with helical flutes of substantially equal or varying pitch along the majority of the length or longitudinal axis.

In some embodiments, the twisted tube, the device tip 208, or portions thereof can be placed inside an outer housing 204. In certain embodiments, the twisted tube and/or the device tip 208 or portions thereof can move axially within the outer housing 204. In certain embodiments, a working length of the twisted tube and/or device tip 208 can be varied by a control system at predetermined oscillating frequencies. For example, the working length can extend about 0 mm, about 1 mm, about 2 mm, about 3 mm, about 4 mm, about 5 mm, about 6 mm, about 7 mm, about 8 mm, about 9 mm, about 10 mm, and/or between a range defined by two of the aforementioned lengths.

The proximal end of the twisted tube can be coupled to a motor or other device configured to generate rotational movement in some embodiments. The motor or other device, in certain embodiments, can drive in one direction, two directions, and/or rotate back and forth in an oscillation motion.

As described above, due to the twisted configuration, as the inner assembly 202, 208 rotates, a pressure gradient can be generated, thereby facilitating the removal of eye tissue that is fragmented by the device tip 208. Such fragmented eye tissue can be aspirated in and/or otherwise removed through the hollow portion of the twisted tube.

Furthermore, in certain embodiments, a proximal end of the twisted tube is coupled to a vacuum source to provide aspiration for removal of tissue material that is fragmented by the device tip 208. In some embodiments, a distal end of the twisted tube coupled to the one or more loops can comprise an opening. Accordingly, the vacuum source has a fluidic connection to the tube through the distal opening. Lens tissue or cataract material that is fragmented by the surgical hand piece can be removed from the surgical area via aspiration.

### Outer Housing

In order to protect the inner housing 202 and/or the device tip 208 and prevent the inner housing 202 and/or device tip 208 from directly contacting undesirable portions within an eye of a patient, it can be advantageous to place the inner housing 202, device tip 208, or portions thereof within an outer housing 204. The outer housing 204 can comprise one or more tubes. In some embodiments, a plastic sleeve can be configured to shield the outer housing 204.

In some embodiments, the inner housing 202 or portions thereof can be configured to be placed inside a first concentric tube or outer housing 204. The outer housing 204 can be configured to closely fit the inner housing 202. In other words, the diameter of the outer housing 204 can be slightly larger than the diameter of the inner housing 202.

In some embodiments, the first tube or outer housing 204 covers substantially the entire inner housing 202. In certain embodiments, a portion of the inner housing 202 and/or device tip 208 protrudes beyond the distal end of the outer housing 204 and is exposed to break up lens tissue or cataract. The device tip 208 protruding from the distal end of the outer housing 204 can comprise a radius slightly larger than the inner diameter of the outer housing 204 such that a constant contact is made between the device tip 208 and the outer housing 204 during operation. A length of the protruding portion of the inner housing 202 can be about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm, about 3.0 mm, or within a range defined by any of the two aforementioned values.

In certain embodiments, a distal end of the outer housing 204 can comprise a loop or forcep-like structure in addition to or in lieu of the device tip 208 of the inner assembly 202, 208. The loop or forcep-like structure of the outer housing 204 can be larger than the device tip 208 of the inner assembly 202, 208. Such loop or forcep-like structure of the first tube can be configured to function in conjunction with the device tip 208 to generate a shearing action as the inner housing 202 and device tip 208 rotate.

Furthermore, in certain embodiments, a proximal end of the outer housing 204 is coupled to a vacuum source to provide suction for removal of tissue material that is fragmented by the device tip 208. In some embodiments, a distal end of the outer housing 204 near the device tip 208 can comprise an opening. Accordingly, aspiration can be generated by the vacuum source and transmitted along the interior of the first tube through the distal opening. Lens tissue or cataract that is fragmented by the device tip 208 can be removed from the surgical area via the aspiration.

In certain embodiments, the outer housing 204 can comprise a fluidic connection to assist aspiration. For example, the outer housing 204 can comprise a shorter second tube fixed concentrically around the first tube. In certain embodiments, the second tube or an infusion sleeve 213 can be configured to provide infusion to the anterior chamber. In some embodiments, the second tube or infusion sleeve 213 can comprise a semi-rigid material. In certain embodiments, the second tube or the infusion sleeve 213 is flared at the base or proximal end that is connected to a casing 206 to create a fluidic connection for the infusion fluid. In some embodiments, an infusion sleeve mount 304 is configured to be positioned at the distal end of the surgical hand piece and attached the infusion sleeve 213 to the casing of the surgical hand piece.

In some embodiments, an interior of the outer housing 204 or one or more tubes thereof can comprise a texture configured to prevent reflux of material from the casing 206 or one or more tubes thereof. In other words, the surface of the interior of the outer housing 204 or one or more tubes thereof can be textured in order to increase traction and remove material more quickly. For example, the interior surface of the outer housing 204 or one or more tubes thereof can comprise a variable texture or can comprise one or more abrading materials of varying surface texture or one or more protrusions.

### Casing

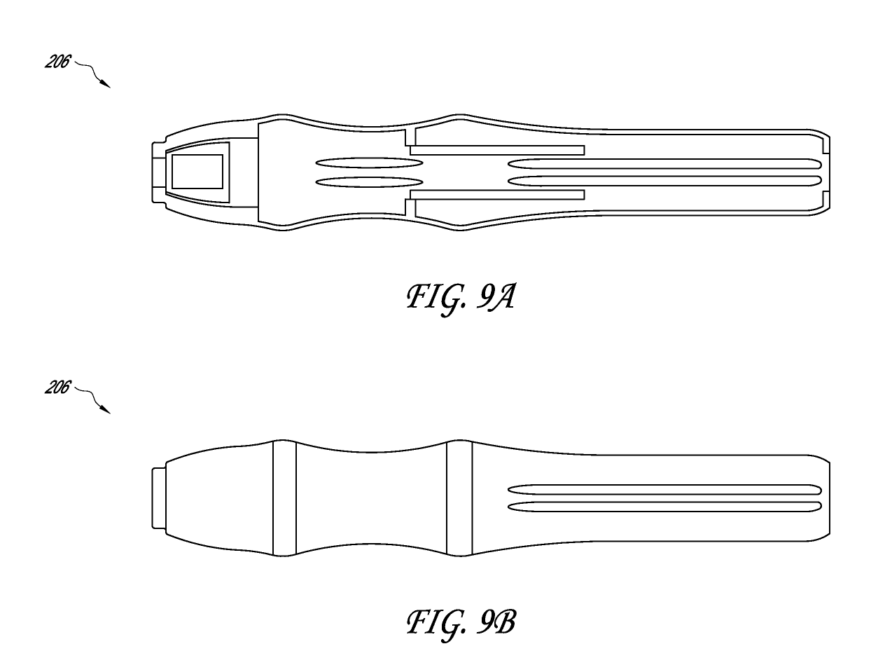

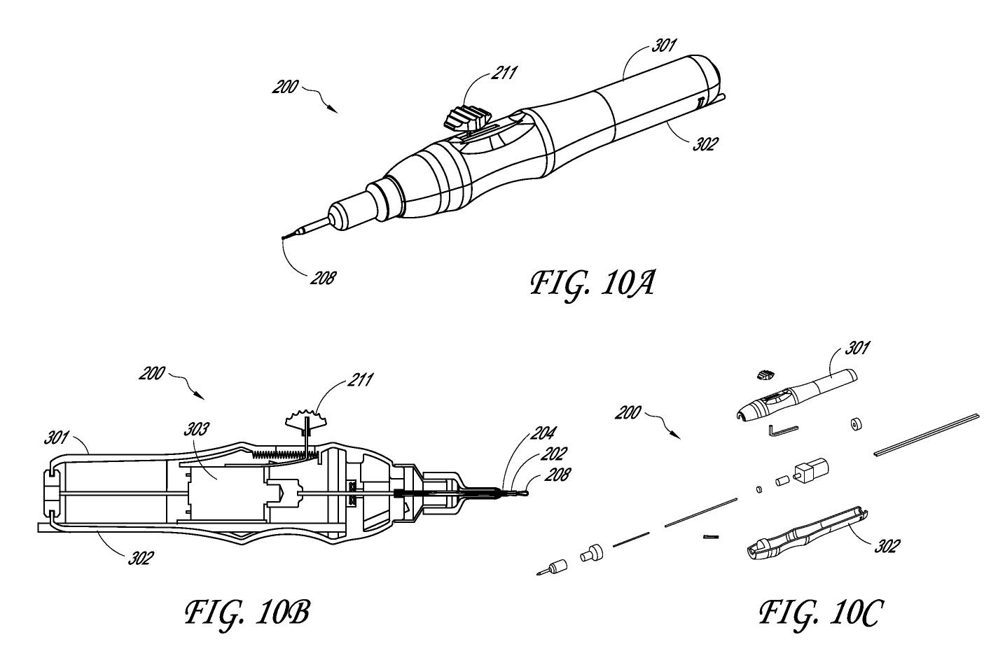

In some embodiments, the outer housing 204, and/or inner assembly 202, 208 can be coupled to a casing 206. FIGS. 9A-B illustrate the casing 206 of the surgical hand piece. FIG. 9A illustrates a top view of the lower section of the casing. FIG. 9B illustrates a bottom view of the lower section of the casing. FIG. 10A illustrates a view of the surgical hand piece casing 301, 302 and the device tip 208 in the extended position. FIG. 10B illustrates a cross section of an embodiment of the surgical hand piece. The surgical hand piece includes the upper and lower casing 301, 302. The casing 206 can include a shell portion with a hollow interior adapted to contain the components of the surgical hand piece. For example, the interior of the casing houses the motor 303 and/or the components that control of the mechanical mechanism 211. The knob of the mechanical mechanism 211 extends out of the casing to allow for the user to manipulate the mechanical mechanism 211. The device tip 208, inner housing 202, and outer housing 204 can be contained in the interior of the casing 206. The end of device tip 208, inner housing 202, and outer housing 204 that is used to contact the patient can extend out of the casing from an opening at one end of the surgical hand piece casing as shown in FIGS. 10A-10B. FIG. 10C illustrates an expanded view of an embodiments of the surgical hand piece. Placement of the inner assembly 202, 208 within a concentric tube or outer housing 204 and attachment to a casing 206 can provide for improved control and manipulation of the surgical hand piece 200 for cataract removal. For example, a proximal end of the outer housing 204 can be fixed to a distal end of the casing 206.

In some embodiments, the casing 206 can comprise a motor, vacuum source or fluidic pathway, and/or infusion source or fluidic pathway, among others. The motor, vacuum source or fluidic pathway, and/or infusion source or fluidic pathway can be located at a distal or proximal end or near the center of the casing 206. For example, the inner assembly 202, 208 can be fixed to a motor within the casing 206 to provide rotation as described herein. In some embodiments, the motor is located near a distal end of the casing 206 such that the motor is located closer to the device tip 208. The motor as described herein can be any device that can drive the movement of the inner assembly and thereby device tip. For example, the inner assembly and/or the device tip can be driven pneumatically, hydraulically, electromechanically, and/or mechanically. The electromechanically driven device can include a motor, a servo, stepper motor, voice coil actuator, solenoid, or any other electromechanical motor that would provide the movement of the inner assembly or device tip. In some embodiments, the mechanically driven device can utilize a rotating cable, torque coil, or any other mechanical device that can provide the movement of the inner assembly or device tip.

A vacuum source or chamber and/or tube can be configured to provide aspiration configured to remove lens material or cataract material that is fragmented by the device tip 208 as described herein. Further, an infusion source or chamber and/or tube can provide infusion fluid and/or fluidic connection for the infusion fluid as described herein.

In some embodiments, the casing 206 comprises controls. In certain embodiments, the controls can be on the exterior surface of the casing 206 and can be configured to allow a surgeon or other user to vary settings of the surgical hand piece 200. For example, in some embodiments, the controls can be configured to vary settings including, but not limited to, rotational speed, aspiration level, infusion level, working length of the inner assembly 202, 208, oscillation frequency of the inner assembly 202, 208, temperature of the internal elements, color of the internal elements, shape of the internal elements, magnetic properties of the internal elements, size of the internal elements, orientation of the internal elements, rotational speed of the internal elements, or the like. In some embodiments, the controls can also be configured to switch between functions.

The casing 206 can comprise any shape or form. For example, in some embodiments, the casing 206 can comprise a substantially cylindrical configuration. In certain embodiments, the casing 206 comprises a conical shape at the distal end.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment. The headings used herein are for the convenience of the reader only and are not meant to limit the scope of the inventions or claims.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. Additionally, the skilled artisan will recognize that any of the above-described methods can be carried out using any appropriate apparatus. Further, the disclosure herein of any particular feature, aspect, method, property, characteristic, quality, attribute, element, or the like in connection with an embodiment can be used in all other embodiments set forth herein. For all of the embodiments described herein the steps of the methods need not be performed sequentially. Thus, it is intended that the scope of the present invention herein disclosed should not be limited by the particular disclosed embodiments described above.

What is claimed is:

1. A device for performing ophthalmic surgery for cataract removal comprising:
a casing;
one or more inner assemblies comprising:
one or more device tips; and
one or more inner housings; and
an outer housing.

2. The device of claim 1, wherein the one or more inner assemblies comprise a rotating element configured to rotate during use of the device.

3. The device of claim 1, wherein the one or more inner assemblies are configured to be coupled to a motor to produce rotational motion.

4. The device of claim 1, wherein the device is used for cataract removal during ophthalmic surgical procedures.

5. The device of claim 4, wherein the device is used to manipulate, cut, fragment, or remove various eye tissues.

6. The device of claim 1, wherein the one or more device tips comprise smooth edges configured to contact a surface of a patient's eye.

7. The device of claim 1, wherein a portion of the one or more inner assemblies is longitudinally surrounded by the outer housing along a length of the one or more inner assemblies.

8. The device of claim 7, wherein the portion of the one or more inner assemblies surrounded by the outer housing is configured to rotate within the outer housing.

9. The device of claim 1, wherein a proximal end of the one or more inner assemblies is configured to be coupled to a distal end of the casing.

10. The device of claim 1, further comprising an aspiration tube and aspiration pathway configured to be in fluid communication with a vacuum source, wherein the vacuum source can aspirate fragmented tissue from a patient's eye.

11. The device of claim 1, further comprising an infusion tube and an infusion pathway configured to provide an infusion of fluid into a surgical area.

12. The device of claim 1, wherein the one or more device tips comprise a biocompatible metal.

13. The device of claim 1, wherein the one or more device tips comprise a material of a smooth texture to minimize traction with surrounding tissues.

14. The device of claim 13, wherein the material of the one or more device tips comprises a coating on the surface of the one or more device tips.

15. The device of claim 1, wherein the one or more device tips comprise a loop configuration.

16. The device of claim 15, wherein the loop configuration comprises more than one loop.

17. The device of claim 15, wherein the loop configuration comprises an off-center loop or non-symmetrical loop shape.

18. The device of claim 1, wherein the one or more device tips comprise a forcep configuration.

19. The device of claim 3, wherein the motor is configured to drive the one or more inner assemblies in one direction, two directions, or rotate back and forth in an oscillation motion.

20. The device of claim 1, wherein the one or more inner housings comprise a flat strip, wherein the flat strip comprises metal twisted into helical flutes.

21. The device of claim 1, wherein the one or more inner housings comprise a hollow rod.

22. The device of claim 1, wherein the one or more inner housings comprise a twisted tube.

23. The device of claim 1, wherein a proximal end of the outer housing is fixed to a distal end of the casing.

24. The device of claim 1, wherein the one or more inner assemblies are configured to translate axially and rotate simultaneously for removal of cataracts.

25. The device of claim 1, wherein the one or more inner housings comprise varying temperatures, wherein the temperature of the one or more inner housings comprise between 0° C. and 50° C.

26. The device of claim 25, wherein the one or more inner housings are configured to be coupled to a heat and/or cooling source configured to vary the temperature of the one or more inner housings.

27. The device of claim 1, wherein the one or more inner housings are configured to be heated and/or cooled during operation to cause further breakdown of the tissue and allow for easier removal.

28. The device of claim 1, further comprising a mechanical mechanism for extending and retracting the one or more inner assemblies.

29. The device of claim 1, wherein the one or more inner assemblies are configured to be coupled to device configured to generate rotational movement, wherein the device configured to generate rotational movement is pneumatically, hydraulically, electromechanically, or mechanically driven.
